(19) United States
(12) Patent Application Publication
(10) Pub. No.: US 2015/0018926 A1
(43) Pub. Date: Jan. 15, 2015

(54) STIMULATION DEVICE

(71) Applicant: cerbomed GmbH, Erlangen (DE)

(72) Inventors: Wolf Gerhard FRENKEL, INZIGKOFEN-ENGELSWIES (DE); Thomas ZSCHAECK, NUERNBERG (DE); Andreas HARTLEP, HOLZKIRCHEN (DE)

(21) Appl. No.: 14/327,129

(22) Filed: Jul. 9, 2014

(30) Foreign Application Priority Data
Jul. 10, 2013 (DE) 10 2013 011 541.8

(51) Int. Cl. A61N 1/04

(52) U.S. Cl. CPC A61N 1/0472; A61N 1/0456
USPC 607/139

(57) ABSTRACT

A stimulation device for transcutaneous electric stimulation stimulus onto the surface of a section of the human ear comprises a holding element attached at or in the ear, at least two electrodes arranged in or at an electrode carrier. To obtain high contact quality of the electrodes with high wearing comfort, the holding element consists of one part designed as holding bow for placement between the surface of the head and the Pinna (P) of the device wearer, the other part designed as connection element between the holding bow and the electrode carrier; between the holding bow and the connection element a hinge joint is arranged such that use of the device allows swivel movement of the connection element relatively to the holding bow around an axis (S) parallel or under an angle (α) of less than 30° to the straight ahead viewing direction (G) of the device wearer.

### CROSS-REFERENCE TO RELATED APPLICATIONS

This application claims the priority of DE 10 2013 011 541.8 filed Jul. 10, 2013, which is incorporated by reference herein.

### BACKGROUND OF THE INVENTION

The invention relates to a stimulation device for the application of a transcutaneous electric stimulation stimulus onto the surface of a section of the human ear, which comprises a holding element which can be attached at or in the ear as well as at least two electrodes which are arranged in or at an electrode carrier.

### SUMMARY OF THE INVENTION

A stimulation device of the generic kind is known from U.S. Pat. No. 5,514,175 A. US 2008/0021517 A1 shows a similar solution.

It is generally known to take influence on the neurophysiological and neuroelectrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

A stimulation device of the kind mentioned above is known from DE 10 2010 054 165 B3. Here, an electrode arrangement is described which comprises a holding element which can be affixed at the ear. At the holding element an electrode carrier is arranged via an elastic section, which electrode carrier holds two electrodes. The electrode carrier consists of an electrical non-conductive plastic material and carries the two electrodes made of metal.

Another design of a stimulation device is known from EP 2 026 872 B1. Here, a stimulation device is described which comprises a housing which can be totally inserted into the Pinna (ear conch). From the housing two bent wire-shaped sections extend, wherein the sections are designed as spring-elastic holders. So, the electrode arrangement can be brought into the required position by gentle clamping in the Pinna so that the ear channel can be charged with a transcutaneous stimulation stimulus.

Although the pre-known stimulation devices deliver already good treatment results certain drawbacks of the devices have turned out in the practice. In this connection the contact quality of the electrodes has to be mentioned which is sometimes not yet optimal, because the skin areas which are reached by the electrodes are not sufficiently big. The increase of the contact pressure onto the skin surface is often no proper measure for the elimination of this difficulty because by doing so the skin of the patient is too much stressed.

Thus, it is an object of the invention, to further develop a stimulation device of the generic kind so that the mentioned drawbacks are eliminated. Thus, a further development has to be proposed so that the contact quality of the electrodes is improved while the skin surface of the patient isn't highly stressed and that a high wearing comfort is obtained.

### DETAILED DESCRIPTION OF THE INVENTION

The solution of this object by the invention is characterized in that the holding element for the electrode carrier consists of two parts, wherein one of the parts is designed as holding bow which is designed for the placement between the surface of the head and the Pinna of the wearer of the stimulation device, wherein the other part is designed as connection element between the holding bow and the electrode carrier, wherein between the holding bow and the connection element a hinge joint is arranged which during intended use of the stimulation device allows a swivel movement of the connection element relatively to the holding bow around an axis which is parallel or under an angle of less than 30° to the straight ahead viewing direction of the wearer (sagittal axis) of the stimulation device.

Insofar the invention proposes a stimulation device with a two-part holding element, wherein one of the parts is hooked behind the ear and the other part holds the electrode carrier with the electrodes. The two said parts are movable hinge joint like, i.e. relatively to another pivotable, wherein the axis of the swivel movement corresponds substantially to the straight ahead viewing direction respectively sagittal direction (it could thus also be said that this direction is directed horizontally forward and perpendicular onto the chest at straight position of the head). The electrode carrier with the electrodes moves at this swivel movement accordingly substantially in transversal direction, i.e. perpendicular to the temporal bone.

With respect to the directions which are relevant here should be mentioned that the straight ahead viewing direction is also called the sagittal direction in the anatomic nomenclature. The horizontal direction perpendicular hereto is the transversal direction; the direction, which is perpendicular to the sagittal direction as well as to the transversal direction is the longitudinal direction (which thus is directed from the head to the feet at a straight standing human).

The hinge joint can thereby be pre-loaded by means of a spring element in a swivel direction. This pre-load can be provided acting in the opening or in the closing direction, i.e. it can move the connection element into the position according to the intended use or away from it. During intentional use the effect of the spring element is used to adjust the contact pressure of the connection element including electrode carrier, which is arranged hinge joint like at the holding bow, in such a manner that the electrode carrier and thus also the electrodes are pressed with low pressure onto the skin surface of the skin area which is to be stimulated.

Preferably, therefore means are provided by which the pre-load of the spring element can be adjusted.

The electrode carrier is preferably rigidly fixed at one end of the connection element, i.e. without the possibility to carry out a relative movement. However, the fixation is preferably designed as releasable fixation, so that the electrode carrier can be taken off. The fixation of the electrode carrier at the connection element can thereby take place via a form-fit connection, especially by means of a dovetail groove, which is machined into the electrode carrier or in the connection element and which cooperates with a congruent counterpart, which is arranged at the connection element or at the electrode carrier.

The holding bow can be designed sickle shaped, while the hinge joint is arranged at one of its ends.

According to a preferred embodiment of the invention, the holding bow can comprise a core made of plastically deformable material, especially of metal. The core can be encased by a casing material, especially by a bio-compatible plastic material, specifically preferred by silicone, by polyamide, by polypropylene or by polyurethane.

The casing material can also consist of a closed-porous foam which nuzzles into the gap between the ear and the cranium. The casing material can comprise at its surface additionally an enhanced adhesion power up to bonding. Thereby an addition hold is given to serve as a bearing for the pre-load of the electrode carrier against the skin of the Cymba. In a preferred embodiment that casing material can be replaceable. The core of the holding bow can also consist of a metal with memory effect (memory function; e.g. Nitinol) which e.g. enhances its bending and thus the pre-load respectively the contact pressure of the electrode carrier against the skin from a temperature of 28° C. In a further embodiment the holding bow itself can equipped with electrodes, e.g. with counter electrodes to the electrodes of the already mentioned electrode carrier. In this case a transmural flow through the tissue with a respective more intensive depth effect of the stimulation is obtained.

The connection element can comprise, at least seen in straight ahead viewing direction of the wearer of the stimulation device, preferably also seen in transversal viewing direction, a substantial L-shaped form. Also, it is preferably relatively to the surface of the head at least so far bent in a convex manner that a contact with the Helix of the ear is prevented which Helix runs out at this location.

The electrode carrier can consist at least partially of elastic material, especially of bio-compatible plastic material, specifically preferred of silicone, of polyethylene, of polypropylene or of polyurethane. The at least two electrodes can thereby at least partially be encased by the material of the electrode carrier. For this partially elastic material the curve of path and pressure is relatively flat. The pressure onto the skin of the Cymba thus rises relatively slow with increasing pre-load, because the electrode carrier becomes broader due to the contact pressure and more and more area is available for the transmittal of the force. This is beneficial for the wearing comfort at the one hand and ensures at the other hand a good contact quality for the application of the stimulation current.

The holding bow can comprise a groove along its bow-shaped run for the placement of an electric cable. But this is only one possibility for the guidance of the cable for the electrical supply of the electrodes. Instead of the groove also other fixation means can be provided, e.g. by means of clips or eyes which are arranged at the stimulation device along the guidance path of the cable (e.g. with a distance of a centimetre in each case).

The electrode carrier comprises, especially designed as electrode head, preferably at least one stimulation electrode and at least one reference electrode.

Preferably, the electrodes are metal electrodes, especially titanium electrodes. They have preferably the shape of a spherical segment or of a section of an ellipsoid.

In a further embodiment the electrode carrier can consist of electrically conductive material or of a material which can be made electrically conductive. In this case it is separated into two sections with different electrical polarity and an isolation section in between.

Preferably, the parts of the stimulation device are interchangeable—as far as skin contact is given—and consist of a soft material, wherein specifically an elastomer material is considered, especially silicone or a material which comprises silicone. In the case of the electrode carrier an easy interchangeability can be obtained in that way that the cable can be plugged into the electrode carrier.

Beneficially, by the proposed design of the electrode device a quite stable construction is obtained without influencing the wearing comfort in a negative way. The electrodes are held stable and reliable in the required position, even then, if accidently a tensile strength is exerted onto the cable leading to the device (e.g. in the case of a head rotation or an arm movement which take with them the cable).

A major part of the otoplastic according to the invention is covered during intentional use by the ear conch, thus the stigmatisation of the wearer is relatively low. Only a part of the connection element is visible which leads along the ending helix of the pinna.

Furthermore, the layout of the design of the otoplastic according to the invention can be made soft and cuddly which grants it an optical and haptical sympathetic impression. An effective strain-relief of the cable is guaranteed by its bent design via almost 270′.

Furthermore, a quick and comfortable possibility for the individual adjustment by selection of different sizes is given by the interchangeability of the components made of a softer material, also a high hygiene standard.

The mentioned hinge joint like slewability is ergonomically beneficial to bring the electrode carrier together with its electrodes in its position in a reliable and easy manner after the holding bow is hooked behind the ear.

A further advantage is that the user can adjust his otoplastic precisely to the anatomy, contour and relief of his own ear and cranium by individual bending and pressing especially of the holding bow.

Furthermore, it is also beneficial that by the proposed design of the stimulation device a good contact quality of the electrodes at the resting on the skin is given without that a high contact pressure of the electrodes on the skin of the patient is necessary.

### BRIEF DESCRIPTION OF THE DRAWINGS

In the drawing an embodiment of the invention is depicted.

FIG. 1 shows the view of an ear with a stimulation device which is arranged at the ear,

FIG. 2 shows the side view of the stimulation device according to FIG. 1, partially shown in a sectional view,

FIG. 3 shows the side view of the lower part of the stimulation device according to FIG. 2,

FIG. 3a shows the section A-B according to FIG. 3 and

FIG. 4 shows the lower part of the stimulation device in view “C” according to FIG. 3.

### DETAILED DESCRIPTION OF THE DRAWINGS

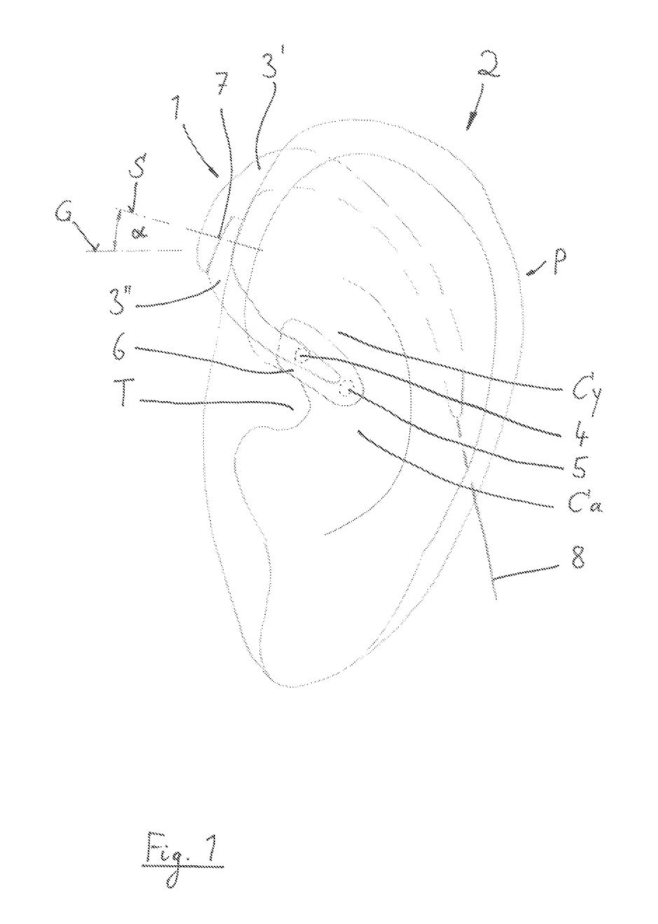

In FIG. 1 an ear 2 of a human is depicted in which a stimulation device 1 is inserted to carry out a transcutaneous electrical stimulation. Hereby, preferably the vagus nerve is subjected to an electrical stimulation to treat different diseases and to obtain effects respectively. Accordingly, with the stimulation device 1 a concrete transcutaneous electrical nerve stimulation can be carried out onto a surface area of the ear. For doing so the stimulation device has a stimulation electrode and a reference electrode (see below), between which an electrical potential is created; the means which are required for doing so are well-known in the state of the art so that they need not be further described here. Exemplary, DE 10 2005 003 735 B4 of the applicant should be mentioned to which explicitly reference is made.

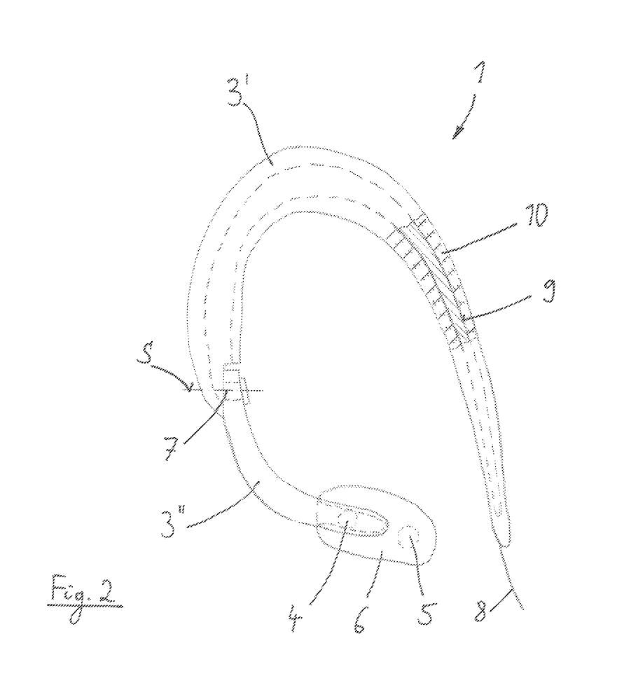

The stimulation device has—as can be further seen in FIGS. 2 to 4—a holding element 3 which carrier an electrode carrier 6. At the electrode carrier 6 two electrodes 4 and 5 are arranged, wherein the electrode 4 is a stimulation electrode and the electrode 5 is a reference electrode. The two electrodes 4, 5 are separated from another by the electrical isolating material of the electrode carrier 6 to create a difference of potential between the electrodes 4, 5. With respect to the design of the stimulation device reference is made explicitly to DE 10 2010 054 165 B3 of the application where this stimulation device is described in detail.

In FIG. 1 the Pinna P of the ear 2 can be seen. The electrode carrier 6 is located in the region of the Cymba conchae Cy; for sake of orientation the Cavum conchae Ca and the tragus T is also depicted.

For the stimulation device 1 the following is essential: The holding element 3 for the electrode carrier 6 consists of two parts, namely of a holding bow 3′ and a connection element 3″. The holding bow 3′ is designed sickle-shaped to be able to be hooked behind the pinna (see FIG. 1), i.e. it is arranged between the surface of the head and the pinna P of the wearer of the stimulation device 1. The connection element 3″ is arranged between the holding bow 3′ and the electrode carrier 6, i.e. it connects those two components of the stimulation device.

Between the holding bow 3′ and the connection element 3″ a hinge joint 7 is arranged. During intentional use of the stimulation device 1, i.e. when the holding bow 3′ is hooked behind the pinna P, a swivel movement of the connection element 3″ relatively to the holding bow 3′ around a swivel axis S is enabled by the hinge joint 7, which swivel axis is substantially directed in straight ahead viewing direction G of the wearer of the stimulation device 1. Thus, at straight standing patient and straight held head this straight ahead viewing direction G would be directed horizontally and perpendicular onto the chest of the patient. However, a small deviation to this direction G can exist insofar in that the swivel axis S has a small angle α of up to 300 to the direction G.

The connection element 3″ can be swiveled against the holding bow 3′ around the swivel axis S in such a manner into the direction of the inner of the head (i.e. in transversal direction) that, with the holding bow 3′ being a counter bearing, a pressure of the electrode carrier 6 can be created against the Cymba Cy.

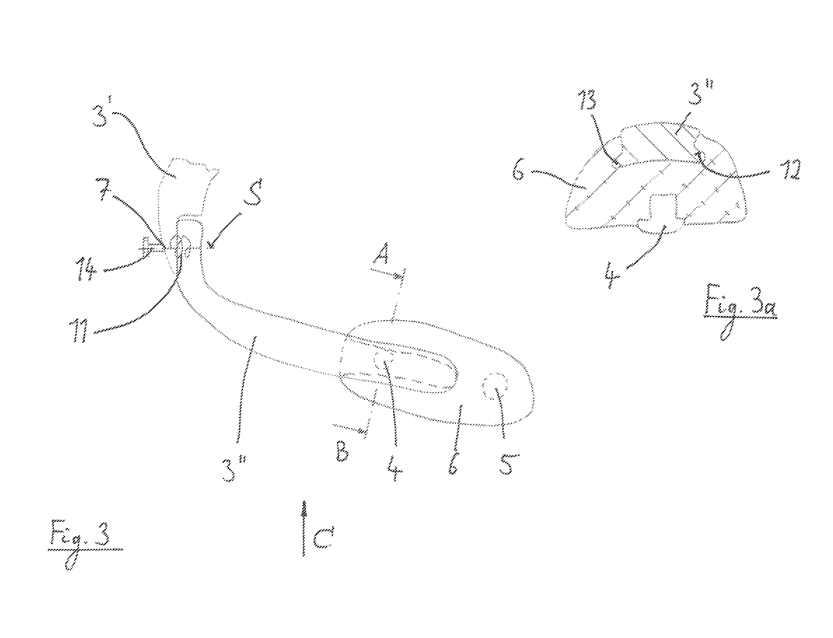

For this purpose a spring element 11 is arranged between the two parts 3′ and 3″ in the region of the hinge joint 7 (see FIG. 3). The spring element 11 ensures that during intentional use of the stimulation device 1 the connection element 3″ with fixed electrode carrier 6 at one end presses with slight pressure onto the skin surface in the region of the Cymba conchae Cy so that—without stressing the skin surface of the wearer of the device too much—a good contact quality of the electrodes 4, 5 on the skin is given. If applicable the electrodes can also be surrounded by porous electrical conductive material to obtain a specifically high wearing comfort.

To adjust the pre-load of the spring element 11 means 14 are provided which are shown only schematically. Those means can e.g. be a knurled screw by which the spring element 11—designed as torsional spring—can be effectively adjusted with its spring effect between the two parts 3′ und 3″

Such a solution allows e.g. also that at first, prior the attachment of the stimulation device at the ear, the connection element 3″ including electrode carrier 6 is swiveled away from the ear until the holding bow 3′ is hooked behind the pinna P. Only then the spring element 11 can be tensed up in such a manner by turning of the knurled screw 14 that the electrode carrier 6 with a pressure is pressed against the skin surface which pressure is convenient.

But it is also possible that the pre-load of the spring element 11 is adjusted by means of the knurled screw 14 so that it corresponds to the later use of the stimulation device 1. Then, the connection element 3″ is held away against the elasticity of the spring element 11 for hooking of the holding bow 3′ behind the pinna P; after release of the connection part 3″ the electrode carrier 6 is laying then with a convenient pre-load on the skin surface in the region of the Cymba conchae Cy.

The electrode carrier 6 is rigid, but preferably interchangeably arranged at the one end of the connection element 3″. For this purpose a form-fit connection according to the key and slot principle is provided, wherein a dovetail groove 12 is arranged which is machined in the electrode carrier 6 (see FIG. 3a). In this groove a congruent shaped end section of the connection element 3″ is meshing with projections 13 as counterpart to fix the electrode carrier 3 rigidly at the connection element 3″.

In FIG. 2 can be seen that the holding bow 3′ consists of a core 9 made of metal which is covered by casing material 10. By this it is possible to make the holding bow 3′ bendable at will to adapt it to the individual desired shape. By the casing—preferably made of bio-compatible soft plastic material—a high wearing comfort is indeed ensured.

The electric cable 8 for the supply of the electrodes 4, 5 with current establishes the connection to a not shown control unit. However, it should be mentioned that also by way of miniaturisation the control unit and as the case may be the energy supply can also be housed in the holding bow 3′.

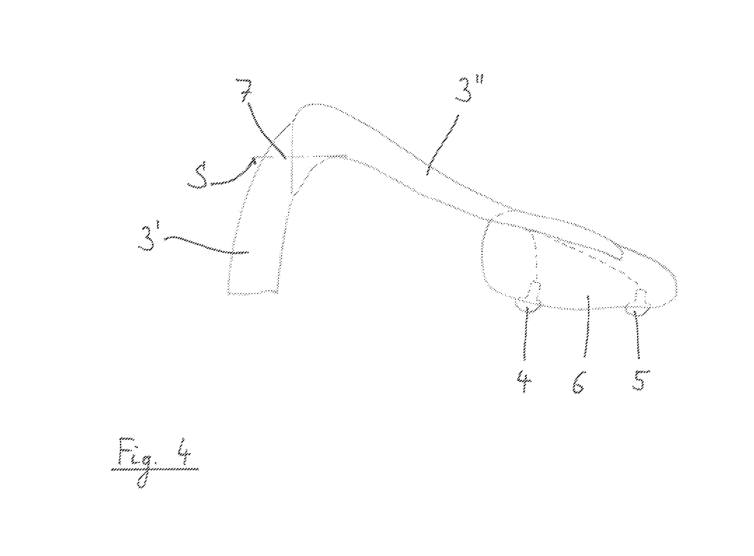

FIG. 4 shows as view C according to FIG. 3 that the holding element 3 with its parts holding bow 3′ and connection element 3″ is bent relatively to the surface of the head at least so far in a convex manner that a contact with the helix of the ear (not shown) is prevented which runs out at this location.

### LIST OF REFERENCES

1 Stimulation device
2 Ear
3 Holding element
3′ Holding bow
3″ Connection element
4 Electrode (stimulation electrode)
5 Electrode (reference electrode)
6 Electrode carrier
7 Hinge joint
8 Electric cable
9 Core (metal insert)
10 Casing material
   11 Spring element
   12 Groove (dovetail groove)
13 Counterpart of the groove (projection)
14 Means for adjusting of the pre-load
S Swivel axis
G Straight ahead viewing direction
α Angle
Ca Cavum conchae
Cy Cymba conchae
T Tragus
P Pinna

1. Stimulation device for the application of a transcutaneous electric stimulation stimulus onto the surface of a section of the human ear, which comprises:
a holding element which can be attached at or in the ear as well as at least two electrodes which are arranged in or at an electrode carrier,
wherein
the holding element for the electrode carrier consists of two parts,
wherein one of the parts is designed as holding bow which is designed for the placement between the surface of the head and the Pinna of the wearer of the stimulation device,
wherein the other part is designed as connection element between the holding bow and the electrode carrier,
wherein between the holding bow and the connection element a hinge joint is arranged which during intended use of the stimulation device allows a swivel movement of the connection element relatively to the holding bow around an axis which is parallel or under an angle of less than 30° to the straight ahead viewing direction of the wearer of the stimulation device.

2. Stimulation device according to claim 1, characterized in that the hinge joint is pre-loaded by means of a spring element in a swivel direction.

3. Stimulation device according to claim 2, characterized in that means are arranged by which the pre-load of the spring element can be adjusted.

4. Stimulation device according to claim 1, characterized in that the electrode carrier is rigidly fixed at one end of the connection element, wherein the fixation is preferably designed as releasable fixation.

5. Stimulation device according to claim 4, characterized in that the fixation of the electrode carrier at the connection element takes place via a form-fit connection, especially by means of a dovetail groove, which is machined into the electrode carrier or in the connection element and which cooperates with a congruent counterpart, which is arranged at the connection element or at the electrode carrier.

6. Stimulation device according to claim 1, characterized in that the holding bow is designed sickle shaped and that the hinge joint is arranged at one of its ends.

7. Stimulation device according to claim 1, characterized in that the holding bow comprises a core made of plastically deformable material, especially of metal, which is encased by a casing material, especially by a bio-compatible plastic material, specifically preferred by silicone, by polyamide, by polypropylene or by polyurethane.

8. Stimulation device according to claim 1, characterized in that the connection element comprises a substantial L-shaped form, seen at least in straight ahead viewing direction of the wearer of the stimulation device, preferably also seen in transversal viewing direction.

9. Stimulation device according to claim 1, characterized in that the electrode carrier consists at least partially of elastic material, especially of bio-compatible plastic material, specifically preferred of silicone, of polyethylene, of polypropylene or of polyurethane, wherein the at least two electrodes are at least partially encased by the material of the electrode carrier.

10. Stimulation device according to claim 1, characterized in that the holding bow comprises a groove along its bow-shaped run for the placement of an electric cable.
